(19) United States
(12) Patent Application Publication
(10) Pub. No.: US 2014/0275576 A1
(43) Pub. Date: Sep. 18, 2014

(54) PROCESS FOR THE PREPARATION OF (2,3-DIHYDRO-BENZO[B][1,4]DIOXIN-2-YL) METHANOL DERIVATIVES

(71) Applicant: Janssen Pharmaceutica NV, Beerse (BE)

(72) Inventor: Hartmut B. Zinser, Schaffhausern (CH)

(73) Assignee: Janssen Pharmaceutica NV, Beerse (BE)

(21) Appl. No.: 14/208,413

(22) Filed: Mar. 13, 2014

Related U.S. Application Data
(60) Provisional application No. 61/779,642, filed on Mar. 13, 2013.

(51) Int. Cl. C07D 319/20

(52) U.S. Cl. CPC C07D 319/20
USPC 549/362

(57) ABSTRACT

The present invention is directed to a process for the preparation of (2,3-dihydrobenzo[b][1,4]dioxin-2-yl)methanol derivatives.

### CROSS REFERENCE TO RELATED APPLICATIONS

This application claims the benefit of U.S. Provisional Application 61/779,642, filed on Mar. 13, 2013, which is incorporated by reference herein in its entirety.

### FIELD OF THE INVENTION

The present invention is directed to a process for the preparation of (2,3-dihydrobenzo[b][1,4]dioxin-2-yl) methanol derivatives.

### BACKGROUND OF THE INVENTION

McComsey, D., et al., in US Patent Publication 2006/00410008 A1, published Feb. 23, 2006 disclose sulfamide derivatives of the following formula (A)

[chemical structure]

useful for the treatment of epilepsy and related disorders. The compounds of formula (A) may be prepared from corresponding alcohol derivatives, compounds of formula (B)

[chemical structure]

as described in, for example McComsey, D., et al. US Patent Publication US 2006/0041008 A1, published Feb. 23, 2006; and McComsey, D., et al. US Patent Publication US 2005/0282887 A1, published Dec. 22, 2005).

Ballentine, S., et al., in U.S. Patent Publication 2009/0247618, published Oct. 1, 2009 teach processes for the preparation of the alcohol derivatives, compounds of formula (B). The process(es) as taught by Ballentine et al., require a lengthy protection-de-protection sequence, and/or high cost starting material(s) and/or high cost reagent(s) and/or oxidation conditions, which make said process(es) unsuitable for large scale/commercial manufacture.

Thus there remains a need for a process for the preparation of compounds of formula (I), and more particularly the compound of formula (I-S), as hereinafter defined, which process is more suitably for commercial or large scale manufacture.

### SUMMARY OF THE INVENTION

The present invention is directed to a process for the preparation of compounds of formula (I)

[chemical structure]

wherein

b is an integer from 0 to 4;

each R⁵ is independently selected from the group consisting of halogen and C₁₋₄alkyl;

and pharmaceutically acceptable salts thereof; comprising

[chemical structure]

reacting a mixture of a compound of formula (V) and a compound of formula (VI), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a base; in an organic solvent or mixture of solvents; at a temperature in the range of from about −10° C. to about 25° C.; to yield a mixture of the corresponding compound of formula (VII-a) and the corresponding compound of formula (VII-b), wherein Cation⁺ is the corresponding base cation;

[chemical structure]

reacting the mixture of the compound of formula (VII-a) and the compound of formula (VII-b) with a compound of formula (IX), an (R)-glycidyl-(substituted aryl)sulfonate, wherein R² is selected from the group consisting of hydrogen, halogen, methyl, ethyl, nitro, cyano, and dichloro; to yield a mixture of the corresponding compound of formula (VIII-a) and the corresponding compound of formula (VIII-b);

[chemical structure]

reacting the mixture of the compound of formula (VIII-a) and the compound of formula (VIII-b) with an aqueous base or a mixture of an aqueous base and an alcohol; in an organic solvent or mixture of organic solvents; at a temperature in the range of from about −10° C. to about 25° C.; to yield the corresponding compound of formula (I).

The present invention is directed to a process for the preparation of compounds of formula (I)

[chemical structure]

wherein

b is an integer from 0 to 4;

each R⁵ is independently selected from the group consisting of halogen and C₁₋₄alkyl;

and pharmaceutically acceptable salts thereof; comprising

[chemical structure]

reacting a mixture of a compound of formula (V) and a compound of formula (VI), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a phosphate; in an organic solvent or mixture of solvents; at about room temperature; to yield a mixture of the corresponding compound of formula (VII-a) and the corresponding compound of formula (VII-b), wherein Cation⁺ is the corresponding phosphate cation;

[chemical structure]

reacting the mixture of the compound of formula (VII-a) and the compound of formula (VII-b) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-a) and the corresponding compound of formula (VIII-b);

[chemical structure]

reacting the mixture of the compound of formula (VIII-a) and the compound of formula (VIII-b) with an aqueous base or a mixture of an aqueous base and an alcohol; in an organic solvent or mixture of organic solvents; at a temperature in the range of from about −10° C. to about 25° C.; to yield the corresponding compound of formula (I).

In an embodiment, the present invention is directed to a process for the preparation of a compound of formula (I-S)

[chemical structure]

or a pharmaceutically acceptable salt thereof; comprising

[chemical structure]

reacting a mixture of a compound of formula (V-S) and a compound of formula (VI-S), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a base; in an organic solvent or mixture of solvents; at a temperature in the range of from about −10° C. to about 25° C.; to yield a mixture of the corresponding compound of formula (VI-Sa) and the corresponding compound of formula (VII-Sb), wherein Cation⁺ is the corresponding base cation;

[chemical structure]

reacting the mixture of the compound of formula (VII-Sa) and the compound of formula (VII-Sb) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-Sa) and the corresponding compound of formula (VIII-Sb);

[chemical structure]

reacting the mixture of the compound of formula (VIII-Sa) and the compound of formula (VIII-Sb) with an aqueous base or a mixture of an aqueous base and an alcohol; in an organic solvent or mixture of organic solvents; at a temperature in the range of from about −10° C. to about 25° C.; to yield the corresponding compound of formula (I-S).

In another embodiment, the present invention is directed to a process for the preparation of a compound of formula (I-S)

[chemical structure]

or a pharmaceutically acceptable salt thereof; comprising

[chemical structure]

reacting a mixture of a compound of formula (V-S) and a compound of formula (VI-S), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a phosphate; at about room temperature; to yield a mixture of the corresponding compound of formula (VI-Sa) and the corresponding compound of formula (VII-Sb), wherein Cation⁺ is the corresponding phosphate cation;

[chemical structure]

reacting the mixture of the compound of formula (VII-Sa) and the compound of formula (VII-Sb) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-Sa) and the corresponding compound of formula (VIII-Sb);

[chemical structure]

reacting the mixture of the compound of formula (VIII-Sa) and the compound of formula (VIII-Sb) with an aqueous base or a mixture of an aqueous base and an alcohol; in an organic solvent or mixture of organic solvents; at a temperature in the range of from about −10° C. to about 25° C.; to yield the corresponding compound of formula (I-S).

The present invention is further directed to a product prepared according to any of the process(es) described herein.

The present invention is further directed to compounds of formula (VII-a), compounds of formula (VII-b), compounds of formula (VIII-a) and compounds of formula (VIII-b), as described in more detail herein, useful as intermediates in the synthesis of the compounds of formula (I). In an embodiment, the present invention is directed to compounds of formula (VII-Sa), compounds of formula (VII-Sb), compounds of formula (VIII-Sa) and compounds of formula (VIII-Sb), as described in more detail herein, useful as intermediates in the synthesis of the compounds of formula (I-S).

### DETAILED DESCRIPTION OF THE INVENTION

The present invention is directed to a process for the preparation of compound of formula (I)

[chemical structure]

wherein b and R⁵ are as herein defined. In an embodiment, the present invention is directed to a process for the preparation of a compound of formula (I-S)

[chemical structure]

or a pharmaceutically acceptable salt thereof. The compounds of formula (I) are useful as intermediates in the synthesis of sulfamide derivatives as disclosed by McComsey et al., in US Patent Publication 2006/00410008 A1, published Feb. 23, 2006.

In an embodiment, the present invention is directed to a process for the preparation of compounds of formula (I) wherein b is an integer from 0 to 2. In another embodiment, the present invention is directed to a process for the preparation of compounds of formula (I) wherein b is an integer from 0 to 1. In another embodiment, the present invention is directed to a process for the preparation of compounds of formula (I) wherein b is an integer from 1 to 2.

In an embodiment, the present invention is directed to a process for the preparation of compounds of formula (I) wherein

[chemical structure]

is selected from the group consisting of 2-(2,3-dihydro-benzo[1,4]dioxinyl), 2-(6-chloro-2,3-dihydro-benzo[1,4]dioxinyl), 2-(6-fluoro-2,3-dihydro-benzo[1,4]dioxinyl), 2-(5-fluoro-2,3-dihydro-benzo[1,4]dioxinyl), 2-(7-chloro-2,3-dihydro-benzo[1,4]dioxinyl), 2-(7-methyl-2,3-dihydro-benzo[1,4]dioxinyl), 2-(5-chloro-2,3-dihydro-benzo[1,4]dioxinyl), 2-(6-bromo-2,3-dihydro-benzo[1,4]dioxinyl), 2-(6,7-dichloro-2,3-dihydro-benzo[1,4]dioxinyl) and 2-(8-chloro-2,3-dihydro-benzo[1,4]dioxinyl).

In another embodiment, the present invention is directed to a process for the preparation of compounds of formula (I) wherein

[chemical structure]

is selected from the group consisting 2-(2,3-dihydro-benzo[1,4]dioxinyl), 2-(6-chloro-2,3-dihydro-benzo[1,4]dioxinyl), 2-(7-chloro-2,3-dihydro-benzo[1,4]dioxinyl), 2-(7-methyl-2,3-dihydro-benzo[1,4]dioxinyl), 2-(6-bromo-2,3-dihydro-benzo[1,4]dioxinyl) and 2-(6,7-dichloro-2,3-dihydro-benzo[1,4]dioxinyl). In another embodiment, the present invention is directed to a process for the preparation of compounds of formula (I) wherein

[chemical structure]

is selected from the group consisting of 2-(2,3-dihydro-benzo[1,4]dioxinyl), 2-(7-methyl-2,3-dihydro-benzo[1,4]dioxinyl) and 2-(6-bromo-2,3-dihydro-benzo[1,4]dioxinyl).

In another embodiment, the present invention is directed to a process for the preparation of compounds of formula (I) wherein

[chemical structure]

is a ring structure selected from the group consisting of 2-(6-chloro-2,3-dihydro-benzo[1,4]dioxinyl), 2-(5-chloro-2,3-dihydro-benzo[1,4]dioxinyl) and 2-(6,7-dichloro-2,3-dihydro-benzo[1,4]dioxinyl). In another embodiment, the present invention is directed to a process for the preparation of compounds of formula (I) wherein

[chemical structure]

is a ring structure selected from the group consisting of 2-(5-chloro-2,3-dihydro-benzo[1,4]dioxinyl) and 2-(6,7-dichloro-2,3-dihydro-benzo[1,4]dioxinyl). In another embodiment, the present invention is directed to a process for the preparation of compounds of formula (I) wherein

[chemical structure]

is 2-(6-chloro-2,3-dihydro-benzo[1,4]dioxinyl).

In an embodiment, the present invention is directed to a process for the preparation of compounds of formula (I) wherein R⁵ is selected from chloro, fluoro, bromo and methyl. In another embodiment, the present invention is directed to a process for the preparation of compounds of formula (I) wherein R⁵ is chloro.

In an embodiment, the present invention is directed to compounds of formula (I) wherein each R⁵ is independently selected from the group consisting of 7-methyl, 6-bromo-, 5-fluoro, 6-fluoro, 5-chloro, 6-chloro, 7-chloro and 8-chloro. In another embodiment, the present invention is directed to compounds of formula (I) wherein each R⁵ is independently selected from the group consisting of 7-methyl, 6-bromo, 6-chloro and 7-chloro.

In another embodiment, the present invention is directed to compounds of formula (I) wherein each R⁵ is independently selected from the group consisting of 7-methyl and 6-bromo. In another embodiment, the present invention is directed to compounds of formula (I) wherein each R⁵ is independently selected from the group consisting of 5-chloro, 6-chloro and 7-chloro.

In another embodiment, the present invention is directed to compounds of formula (I) wherein b is an integer from 0 to 1 and R⁵ is 6-chloro. In another embodiment, the present invention is directed to a compound of formula (I) wherein R⁵ is 6-chloro.

In an embodiment, the present invention is directed to a process for the preparation of compounds of formula (I) wherein the stereo-center on the compound of formula (I) is in the S-configuration. In another embodiment, the present invention is directed to a process for the preparation of compounds of formula (I) wherein the stereo-center on the compound of formula (I) is in the R-configuration.

In an embodiment, the present invention is directed to a process for the preparation of compounds of formula (I) wherein the compound of formula (I) is present as an enantiomerically enriched mixture, wherein the % enantiomeric enrichment (% ee) is greater than about 75%, preferably greater than about 90%, more preferably greater than about 95%, most preferably greater than about 98%. In an embodiment, the present invention is directed to a process for the preparation of compounds of formula (I-S) wherein the compound of formula (I-S) is present as an enantiomerically enriched mixture, wherein the % enantiomeric enrichment (% ee) is greater than about 75%, preferably greater than about 90%, more preferably greater than about 95%, most preferably greater than about 98%.

As used herein, unless otherwise noted, “halogen” shall mean chlorine, bromine, fluorine and iodine.

As used herein, unless otherwise noted, the term “alkyl” whether used alone or as part of a substituent group, includes straight and branched chains. For example, alkyl radicals include methyl, ethyl, propyl, isopropyl, butyl, isobutyl, sec-butyl, t-butyl, pentyl and the like. Unless otherwise noted, the term “C_{X-Y}alkyl” wherein X and Y are integers shall include straight and branched chain composition of between X and Y carbon atoms. For example, “C₁₋₄alkyl” shall include straight and branched chain composition of between 1 and 4 carbon atoms, including methyl, ethyl, n-propyl, isopropyl, n-butyl, isobutyl and t-butyl.

As used herein, the term “alkenyl” whether used alone or as part of a substituent group, include straight and branched carbon chains containing at least one double bond, preferably one double bond. Unless otherwise noted, the term “C_{X-Y}alkenyl” wherein X and Y are integers shall include straight and branched chain composition containing at least one double bond of between X and Y carbon atoms. For example, “C₂₋₄alkenyl” shall include straight and branched chain composition of between 2 and 4 carbon atoms containing at least one double bong, including for example, —CH═CH₂, —CH₂—CH═CH₂, —CH═CH—CH₃, —CH₂—CH₂—CH═CH₂, —CH₂—CH═CH—CH₃, —CH═CH—CH₂—CH₃, and the like.

As used herein, unless otherwise noted, “aryl” shall refer to unsubstituted carbocylic aromatic groups such as phenyl, naphthyl, and the like, preferably phenyl.

As used herein, unless otherwise noted, the term “substituted aryl” shall refer to a carbocyclic aromatic group as herein defined, preferably phenyl, wherein the carbocyclic aromatic group is substituted with one or more, preferably one to three, more preferably one to two independently selected substituents. Suitably substituents include, but are not limited to halogen, C₁₋₄alkyl, cyano, nitro, di(C1-4alkyl)amino, carboxamido, and the like.

As used herein, unless otherwise noted, “aralkyl” shall mean any lower alkyl group substituted with an aryl group such as phenyl, naphthyl and the like. For example, benzyl, phenylethyl, phenylpropyl, naphthylmethyl, and the like, preferably benzyl or phenylethyl.

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure.

Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

As used herein, the notation “*” shall denote the presence of a stereogenic center.

When a particular group is “substituted” (e.g., alkyl, aryl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

[chemical structure]

Abbreviations used in the specification, particularly the Schemes and Examples, are as follows:

DBU=2,3,4,6,7,8,9,10-Octahydropyrimido[1,2-a]azepine

DIPEA or DIEA=Diisopropylethylamine

DMA=Dimethylacetamide

DMF=N,N-Dimethylformamide

DMSO=Dimethylsulfoxide

EtOH=Ethanol

HPLC=High Pressure Liquid Chromatography

IPA=Isopropyl alcohol

KOtBu=Potassium tert-Butoxide

LiOtBu=Lithium tert-Butoxide

MTBE or TBME=Methyl tert-Butyl Ether

TFA=Trifluoroacetic Acid

THF=Tetrahydrofuran

As used herein, unless otherwise noted, the term “isolated form” shall mean that the compound is present in a form which is separate from any solid mixture with another compound(s), solvent system or biological environment. In an embodiment, the present invention is directed to a process wherein the compound of formula (I) is prepared in an isolated form. In an embodiment, the present invention is directed to a process wherein the compound of formula (I-S) is prepared in an isolated form.

As used herein, unless otherwise noted, the term “substantially pure compound” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment, the present invention is directed to a process wherein the compound of formula (I) is prepared as a substantially pure compound. In an embodiment, the present invention is directed to a process wherein the compound of formula (I-S) is prepared as a substantially pure compound.

In another embodiment, the present invention is directed to a product prepared according to any of the processes described herein, wherein the product is substantially pure. In another embodiment, the present invention is directed to a product prepared according to any of the processes described herein, wherein the product is a substantially pure form of the compound of formula (I-S).

As used herein, unless otherwise noted, the term “substantially free of a corresponding salt form(s)” when used to describe the compound of formula (I) shall mean that mole percent of the corresponding salt form(s) in the isolated compound of formula (I) is less than about 5 mole percent, preferably less than about 2 mole percent, more preferably, less than about 0.5 mole percent, most preferably less than about 0.1 mole percent. In an embodiment, the present invention is directed to a process wherein the compound of formula (I) is prepared in a form which is substantially free of corresponding salt form(s). In an embodiment, the present invention is directed to a process wherein the compound of formula (I-S) is prepared in a form which is substantially free of corresponding salt form(s).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α-obs]/[α-max])×100.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, 1,1-dichloroethane, dichloromethane, MTBE, toluene, acetone, and the like.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### GENERAL SYNTHESIS SCHEMES

The present invention is directed to a process for the preparation of compounds of formula (I), as outlined in more detail in Scheme 1, below.

[chemical structure]

[chemical structure]

Accordingly, a mixture of a suitably substituted compound of formula (V), a known compound or compound prepared by known methods, and a suitably substituted compound of formula (VI), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, preferably, R¹ is selected from the group consisting of C₁₋₄alkyl, substituted phenyl, —(C₁₋₂alkyl)-phenyl, heteroaryl and —O—C₁₋₄alkyl, for example, R¹ is methyl, ethyl, isopropyl, n-butyl, isobutyl, t-butyl, allyl, benzyl, 2,2,2-trichloroethyl, and the like, preferably, —O—C(O)—O-isobutyl, and the like, (and wherein R¹ is preferably other than phenyl, nitrophenyl and imidazolyl), a known compound or compound prepared by known methods;

wherein the compound of (VI) is preferably present in an amount in the range of from about 1.0 to about 1.5 molar equivalents (relative to the moles of the compound of formula (V-S)), preferably in an amount in the range of from about 1.05 to about 1.2 molar equivalents, for example, in an amount in the range of from about 1.1 to about 1.2 molar equivalents;

is reacted with a suitably selected base such as DBU, KOtBu, LiOtBu, NaOC₅H₁₁, Cs₂CO₃, Na₂CO₃, K₂CO₃, and the like, preferably, a base such as DBU, KOtBu, LiOtBu, NaOC₅H₁₁, Cs₂CO₃, more preferably, an organic base such as DBU, KOtBu, LiOtBu, and the like, more preferably KOtBu; wherein the base is preferably present in an amount in the range of from about 1.0 to about 3.0 molar equivalents (relative to the moles of the compound of formula (V)), preferably in an amount in the range of from about 1.75 to about 2.25 molar equivalents, for example in an amount in the range of from about 2.1 to about 2.5 molar equivalents;

in a suitably selected organic solvent or mixture of organic solvents such as DMA, toluene, DMF, acetonitrile, THF, 1,4-dioxane, MTBE, and the like, preferably in a dipolar-aprotic organic solvent such as DMA, DMF, acetonitrile, and the like, for example in DMA; at a temperature in the range of from about −10° C. to about 25° C., preferably at a temperature in the range of from about −5° C. to about 10° C., more preferably at a temperature in the range of from about 0° C. to about 5° C.; to yield a mixture of the corresponding mono-acylated compounds, i.e. a mixture of the corresponding compound of formula (VII-a) and the corresponding compound of formula (VII-b), wherein the Cation⁺ is the corresponding base cation. For example, wherein the base is KOtBu, the Cation⁺ is K⁺, wherein the base is LiOtBu, the Cation⁺ is Li⁺, wherein the base is DBU, the Cation⁺ is

[chemical structure]

wherein the base is NaOC₅H₁₁, the Cation⁺ is Na⁺, or wherein the base is Cs₂CO₃, the Cation⁺ is Cs⁺².

The mixture of the compound of formula (VII-a) and the compound of formula (VII-b) is preferably not isolated or purified.

One skilled in the art will recognize that wherein the compound of formula (IV), R¹ is selected from the group consisting of C₁₋₄alkyl, C₂₋₄alkenyl, substituted aryl, aralkyl and heteroaryl, then —C(O)—R¹ is a suitably selected acyl group such as —C(O)—CH₃ (i.e. acetyl, wherein R¹ is —CH₃), —C(O)C(CH₃)₃ (i.e. pivaloyl, wherein R¹ is —C(CH₃)₃), —C(O)-phenyl (i.e. benzoyl, wherein R¹ is phenyl), and the like (and wherein —C(O)—R¹ is other than —C(O)H (i.e. R¹ is not H)); alternatively, wherein the compound of formula (IV) R¹ is selected from the group consisting of —O—C₁₋₄alkyl, —O—C₂₋₄alkenyl, —O— (substituted aryl), —O-aralkyl and —O-heteroaryl, then —O—C(O)—O—R¹ is a suitably selected carbonate group, such as for example —O—C(O)—O-isobutyl, and the like.

Alternatively, a mixture of a suitably substituted compound of formula (V), a known compound or compound prepared by known methods, and a suitably substituted compound of formula (VI), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-alkyl and —O-heteroaryl, preferably, R¹ is selected from the group consisting of C₁₋₄alkyl, substituted phenyl, —C₁₋₂alkyl-phenyl, heteroaryl and —O—C₁₋₄alkyl, for example, R¹ is methyl, ethyl, isopropyl, n-butyl, isobutyl, t-butyl, allyl, benzyl, 2,2,2-trichloroethyl, and the like, preferably, —O—C(O)—O-isobutyl, and the like (and wherein R¹ is preferably other than phenyl, nitrophenyl and imidazolyl), a known compound or compound prepared by known methods;

wherein the compound of (VI) is preferably present in an amount in the range of from about 1.0 to about 1.5 molar equivalents (relative to the moles of the compound of formula (V)), preferably in an amount in the range of from about 1.05 to about 1.2 molar equivalents, for example, in an amount in the range of from about 1.1 to about 1.2 molar equivalents;

is reacted with a suitably selected phosphate such as potassium phosphate, sodium phosphate, and the like; wherein the phosphate is preferably present in an amount in the range of from about 2.0 to about 10.0 molar equivalents (relative to the moles of the compound of formula (V)), preferably in an amount in the range of from about 2.0 to about 8.0 molar equivalents, for example in an amount of about 6.0 molar equivalents; in a suitably selected organic solvent or mixture of organic solvents such as DMA, toluene, DMF, acetonitrile, THF, 1,4-dioxane, MTBE, and the like, preferably in a dipolar-aprotic organic solvent such as DMA, DMF, acetonitrile, and the like, for example in DMA; preferably at about room temperature; to yield a mixture of the corresponding mono-acylated compounds, i.e. a mixture of the corresponding compound of formula (VII-a) and the corresponding compound of formula (VII-b), wherein the Cation⁺ is the corresponding phosphate cation. For example, wherein the phosphate is potassium phosphate, then the Cation⁺ is K⁺; wherein the phosphate is sodium phosphate, then the Cation⁺ is Na⁺.

One skilled in the art will recognize that reacting the mixture of the compound of formula (V) and the compound of formula (VI) with the base results in an equilibrium with the resulting mixture of the compound of formula (VII-a) and the compound of formula (VII-b); and that the base, solvent, temperature and other reaction condition(s) are preferably selected to drive the equilibrium towards formation of the mixture of mono-acylated compounds of formula (VII-a) and (VII-b).

The mixture of the compound of formula (VII-a) and the compound of formula (VII-b) is reacted with a suitably substituted (R)-glycidyl-(substituted aryl)-sulfonate, a compound of formula (IX) (wherein R² is a suitably selected aryl substituent group such as hydrogen, halogen, methyl, ethyl, nitro, cyano, dichloro, and the like (for example, wherein R² is 3-methyl, 4-methyl, 4-ethyl, 2-halo, 3-halo, 4-halo, 2-nitro, 3-nitro, 4-nitro, 4-cyano, and the like), and the like), preferably, R² is selected from the group consisting of hydrogen, halogen, methyl, ethyl, nitro and cyano, more preferably the compound of formula (IX) is for example, (R)-glycidyl-3-nitrobenzenesulfonate (also known as (S)-oxiran-2-ylmethyl 3-nitrobenzenesulfonate), (R)-glycidyl-4-nitrobenzenesulfonate, (R)-glycyl-4-tolylsulfonate, and the like, a known compound or compound prepared by known methods; more preferably, the compound of formula (IX) is (R)-glycidyl-3-nitrobenzenesulfonate, and is added to the reaction mixture as a solid;

wherein the compound of formula (IX) (for example, (R)-glycidyl-3-nitrobenzenesulfonate) is preferably present in an amount in the range of from about 0.2 to about 3.0 molar equivalents (relative to the moles of the compound of formula (V)), preferably in an amount in the range of from about 0.5 to about 1.5 molar equivalents, for example, in an amount of about 0.9 molar equivalents; to yield a mixture of the corresponding compound of formula (VIII-a) and the corresponding compound of formula (VIII-b).

Preferably, the suitably substituted (R)-glycidyl-(substituted aryl)-sulfonate compound of formula (IX) (for example (R)-glycidyl-3-nitrobenzenesulfonate) is added directly to the mixture resulting from the reaction of the mixture of the compound of formula (V) and the compound of formula (VI) with the base or phosphate, preferably with the base.

One skilled in the art will recognize that the suitably substituted (R)-glycidyl-(substituted aryl)-sulfonate compound of formula (IX) is selected to provide a source of

[chemical structure]

The mixture of the compound of formula (VIII-a) and the compound of formula (VIII-b) is reacted a suitably selected aqueous base such as NaOH, KOH, LiOH, and the like or a mixture of a suitably selected base and a suitably selected alcohol such as a mixture of NaOH or KOH in methanol, a mixture of NaOH or KOH in ethanol, NaOH or KOH in isopropanol, and the like; wherein the aqueous base is preferably present in an amount in the range of from about 2.0 to about 8.0 molar equivalents (relative to the moles of the mixture of the compound of formula (VIII-a) and the compound of formula (VIII-b)), preferably present in an amount in the range of from about 3.0 to about 5.0 molar equivalents, for example in an amount of about 4.0 molar equivalents;

at a temperature in the range of from about −10° C. to about 25° C., preferably at a temperature in the range of from about −5° C. to about 10° C., more preferably at a temperature in the range of from about 0° C. to about 5° C.; to yield the corresponding compound of formula (I).

Preferably, the compound of formula (I) is further isolated and/or purified according to known methods. In an example, the compound of formula (I) is isolated by filtration. In another example, the compound of formula (I) is purified by recrystallization.

In an embodiment, the present invention is directed to a process for the preparation of compounds of formula (I-S), as outlined in more detail in Scheme 2, below.

[chemical structure]

[chemical structure]

Accordingly, a mixture of a suitably substituted compound of formula (V-S), a known compound or compound prepared by known methods, and a suitably substituted compound of formula (VI-S), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, preferably, R¹ is selected from the group consisting of C₁₋₄alkyl, substituted phenyl, —C₁₋₂alkyl-phenyl, heteroaryl and —O—C₁₋₄alkyl, for example, R¹ is methyl, ethyl, isopropyl, n-butyl, isobutyl, t-butyl, allyl, benzyl, 2,2,2-trichloroethyl, and the like, preferably, —O—C(O)—O-isobutyl, and the like, (and wherein R¹ is preferably other than phenyl, nitrophenyl and imidazolyl), a known compound or compound prepared by known methods;

wherein the compound of (VI-S) is preferably present in an amount in the range of from about 1.0 to about 1.5 molar equivalents (relative to the moles of the compound of formula (V-S)), preferably in an amount in the range of from about 1.05 to about 1.2 molar equivalents, for example, in an amount in the range of from about 1.1 to about 1.2 molar equivalents;

is reacted with a suitably selected base such as DBU, KOtBu, LiOtBu, NaOC₅H₁₁, Cs₂CO₃, Na₂CO₃, K₂CO₃, and the like, preferably, a base such as DBU, KOtBu, LiOtBu, NaOC₅H₁₁, Cs₂CO₃, more preferably, an organic base such as DBU, KOtBu, LiOtBu, and the like, more preferably KOtBu; wherein the base is preferably present in an amount in the range of from about 1.0 to about 3.0 molar equivalents (relative to the moles of the compound of formula (V-S)), preferably in an amount in the range of from about 1.75 to about 2.25 molar equivalents, for example in an amount in the range of from about 2.1 to about 2.5 molar equivalents;

in a suitably selected organic solvent or mixture of organic solvents such as DMA, toluene, DMF, acetonitrile, THF, 1,4-dioxane, MTBE, and the like, preferably in a dipolar-aprotic organic solvent such as DMA, DMF, acetonitrile, and the like, for example in DMA; at a temperature in the range of from about −10° C. to about 25° C., preferably at a temperature in the range of from about −5° C. to about 10° C., more preferably at a temperature in the range of from about 0° C. to about 5° C.; to yield a mixture of the corresponding mono-acylated compounds, i.e. a mixture of the corresponding compound of formula (VII-Sa) and the corresponding compound of formula (VII-Sb), wherein the Cation⁺ is the corresponding base cation. For example, wherein the base is KOtBu, the Cation⁺ is K⁺, wherein the base is LiOtBu, the Cation⁺ is Li⁺, wherein the base is DBU, the Cation⁺ is

[chemical structure]

wherein the base is NaOC₅H₁₁, the Cation⁺ is Na⁺, or wherein the base is Cs₂CO₃, the Cation⁺ is Cs⁺².

The mixture of the compound of formula (VII-Sa) and the compound of formula (VII-Sb) is preferably not isolated or purified.

One skilled in the art will recognize that wherein the compound of formula (IV-S), R¹ is selected from the group consisting of C₁₋₄alkyl, C₂₋₄alkenyl, substituted aryl, aralkyl and heteroaryl, then —C(O)—R¹ is a suitably selected acyl group such as —C(O)—CH₃ (i.e. acetyl, wherein R¹ is —CH₃), —C(O)C(CH₃)₃ (i.e. pivaloyl, wherein R¹ is —C(CH₃)₃), —C(O)-phenyl (i.e. benzoyl, wherein R¹ is phenyl), and the like (and wherein —C(O)—R¹ is other than —C(O)H (i.e. R¹ is not H)); alternatively, wherein the compound of formula (IV-S) R¹ is selected from the group consisting of, —O—C₁₋₄alkyl, —O—C₂₋₄alkenyl, —O— (substituted aryl), —O— aralkyl and —O-heteroaryl, then —O—C(O)—O—R¹ is a suitably selected carbonate group, such as for example —O—C(O)—O-isobutyl, and the like.

Alternatively, a mixture of a suitably substituted compound of formula (V-S), a known compound or compound prepared by known methods, and a suitably substituted compound of formula (VI-S), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, preferably, R¹ is selected from the group consisting of C₁₋₄alkyl, substituted phenyl, —C₁₋₂alkyl-phenyl, heteroaryl and —O—C₁₋₄alkyl, for example, R¹ is methyl, ethyl, isopropyl, n-butyl, isobutyl, t-butyl, allyl, benzyl, 2,2,2-trichloroethyl, and the like, preferably, —O—C(O)—O-isobutyl, and the like (and wherein R¹ is preferably other than phenyl, nitrophenyl and imidazolyl), a known compound or compound prepared by known methods;

wherein the compound of (VI-S) is preferably present in an amount in the range of from about 1.0 to about 1.5 molar equivalents (relative to the moles of the compound of formula (V-S)), preferably in an amount in the range of from about 1.05 to about 1.2 molar equivalents, for example, in an amount in the range of from about 1.1 to about 1.2 molar equivalents;

is reacted with a suitably selected phosphate such as potassium phosphate, sodium phosphate, and the like; wherein the phosphate is preferably present in an amount in the range of from about 2.0 to about 10.0 molar equivalents (relative to the moles of the compound of formula (V-S)), preferably in an amount in the range of from about 2.0 to about 8.0 molar equivalents, for example in an amount of about 6.0 molar equivalents;

in a suitably selected organic solvent or mixture of organic solvents such as DMA, toluene, DMF, acetonitrile, THF, 1,4-dioxane, MTBE, and the like, preferably in a dipolar-aprotic organic solvent such as DMA, DMF, acetonitrile, and the like, for example in DMA; preferably at about room temperature; to yield a mixture of the corresponding mono-acylated compounds, i.e. a mixture of the corresponding compound of formula (VII-Sa) and the corresponding compound of formula (VII-Sb), wherein the Cation⁺ is the corresponding phosphate cation. For example, wherein the phosphate is potassium phosphate, then the Cation⁺ is K⁺; wherein the phosphate is sodium phosphate, then the Cation⁺ is Na⁺.

One skilled in the art will recognize that reacting the mixture of the compound of formula (V-S) and the compound of formula (VI-S) with the base results in an equilibrium with the resulting mixture of the compound of formula (VII-Sa) and the compound of formula (VII-Sb); and that the base, solvent, temperature and other reaction condition(s) are preferably selected to drive the equilibrium towards formation of the mixture of mono-acylated compounds of formula (VII-Sa) and (VII-Sb).

The mixture of the compound of formula (VII-Sa) and the compound of formula (VII-Sb) is reacted with a suitably substituted (R)-glycidyl-(substituted aryl)-sulfonate, a compound of formula (IX) (wherein R² is a suitably selected aryl substituent group such as hydrogen, halogen, methyl, ethyl, nitro, cyano, dichloro, and the like, (for example, wherein R² is 3-methyl, 4-methyl, 4-ethyl, 2-halo, 3-halo, 4-halo, 2-nitro, 3-nitro, 4-nitro, 4-cyano, and the like), and the like), preferably, R² is selected from the group consisting of hydrogen, halogen, methyl, ethyl, nitro and cyano, more preferably the compound of formula (IX) is for example, (R)-glycidyl-3-nitrobenzenesulfonate (also known as (S)-oxiran-2-ylmethyl 3-nitrobenzenesulfonate), (R)-glycidyl-4-nitrobenzenesulfonate, (R)-glycyl-4-tolylsulfonate, and the like, a known compound or compound prepared by known methods; more preferably, the compound of formula (IX) is (R)-glycidyl-3-nitrobenzenesulfonate, and is added to the reaction mixture as a solid;

wherein the compound of formula (IX) (for example, (R)-glycidyl-3-nitrobenzenesulfonate) is preferably present in an amount in the range of from about 0.2 to about 3.0 molar equivalents (relative to the moles of the compound of formula (V-S)), preferably in an amount in the range of from about 0.5 to about 1.5 molar equivalents, for example, in an amount of about 0.9 molar equivalents; to yield a mixture of the corresponding compound of formula (VIII-Sa) and the corresponding compound of formula (VIII-Sb).

Preferably, the suitably substituted (R)-glycidyl-(substituted aryl)-sulfonate compound of formula (IX) (for example (R)-glycidyl-3-nitrobenzenesulfonate) is added directly to the mixture resulting from the reaction of the mixture of the compound of formula (V-S) and the compound of formula (VI-S) with the base or phosphate, preferably with the base.

One skilled in the art will recognize that the suitably substituted (R)-glycidyl-(substituted aryl)-sulfonate compound of formula (IX) is selected to provide a source of

[chemical structure]

The mixture of the compound of formula (VIII-Sa) and the compound of formula (VIII-Sb) is reacted a suitably selected aqueous base such as NaOH, KOH, LiOH, and the like or a mixture of a suitably selected base and a suitably selected alcohol such as a mixture of NaOH or KOH in methanol, a mixture of NaOH or KOH in ethanol, NaOH or KOH in isopropanol, and the like; wherein the aqueous base is preferably present in an amount in the range of from about 2.0 to about 8.0 molar equivalents (relative to the moles of the mixture of the compound of formula (VIII-Sa) and the compound of formula (VIII-Sb)), preferably present in an amount in the range of from about 3.0 to about 5.0 molar equivalents, for example in an amount of about 4.0 molar equivalents;

at a temperature in the range of from about −10° C. to about 25° C., preferably at a temperature in the range of from about −5° C. to about 10° C., more preferably at a temperature in the range of from about 0° C. to about 5° C.; to yield the corresponding compound of formula (I-S).

The compound of formula (I-S) is preferably further isolated and/or purified, according to known methods as would be readily recognized by one skilled in the art. For example, the compound of formula (I-S) is isolated by aqueous work-up and/or filtration. Preferably, the compound of formula (I-S) is crystallized from a suitably selected solvent such as cyclohexane, and the like.

One skilled in the art will recognize that compounds of formula (VI) may be prepared from the corresponding compound of formula (V), by reacting with a suitably selected acylating agent such as a suitably substituted acyl halide (e.g. acyl chloride), a suitably substituted anhydride, and the like, according to known methods. For example, wherein —C(O)—R¹ is acetyl, the compound of formula (V) may be reacted with greater than about 2 molar equivalents, for example with about 2.1 molar equivalents of for example, acetic acid anhydride; alternatively, wherein —C(O)—R¹ is benzoyl, the compound of formula (V) may be reacted with greater than about 2 molar equivalents, for example with about 2.1 molar equivalents of for example, benzoic acid chloride; alternatively, wherein —C(O)—R¹ is pivaloyl, the compound of formula (V) may be reacted with greater than about 2 molar equivalents, for example with about 2.1 molar equivalents of for example, pivaloyl chloride; in the presence of a suitably organic base such as TEA, DIPEA, pyridine, and the like; in a suitably selected organic solvent such as THF, TBME, toulene, and the like.

One skilled in the art will recognize that in the processes as described in Schemes 1 and 2, above, when the compound of formula (V) is substituted with one to two R⁵ substituents (i.e. wherein b is an integer from 1 to 2) then the cyclization of the mixture of the compound of formula (VIII-a) and the compound of formula (VIII-b) results in a mixture of two regioisomers—a desired regioisomer and a by-product regioisomer. One skilled in the art will further recognize that the reaction condition(s) are preferably selected to maximize the yield of the desired regioisomer, and minimize formation of the undesired or by-product regioisomer.

For example, in the process as described in Scheme 2, above, cyclization of the mixture of the compound of formula (VIII-Sa) and the compound of formula (VIII-Sb) results in a mixture of two regioisomers—a desired regioisomer, the compound of formula (I-S) and an undesired/by-product regioisomer, a compound of the formula (I-T)

[chemical structure]

also known as (S)-(7-chloro-2,3-dihydrobenzo[b][1,4]dioxin-2-yl)methanol. Preferably, the reaction condition(s) are selected to maximize the yield of the desired compound of formula (I-S), and minimize formation of the compound of formula (I-T).

One skilled in the art will further recognize that the undesired/by-product regioisomer is a characteristic impurity or by-product of the process of the present invention. For example, in the process for the preparation of the compound of formula (I-S), as described in Scheme 2 above, the compound of formula (I-T) is a characteristic impurity or by-product of said process.

One skilled in the art will further recognize that the presence of the undesired/by-product regioisomer in a product prepared according to the process of the present invention is characteristic of the use of the process of the present invention. For example, wherein the compound of formula (I-S) is prepared as a product of the process of the present invention, the presence of the compound of formula (I-T) in the product of said process is characteristic of the use of said process of the present invention.

In an embodiment, the present invention is directed to a process for the preparation of the compound of formula (I), as described above, wherein the undesired regioisomer is present in an amount less than about 10 mole percent, preferably less than about 8 mole %, more preferably less than about 5 mole %, more preferably less than or equal to about 4 mole %, more preferably less than about 2 mole %, more preferably less than about 1.5 mole %, more preferably less than about 1 mole %.

In an embodiment, the present invention is directed to a process for the preparation of the compound of formula (I-S), as described above, wherein the undesired regioisomer, the compound of formula (I-T) is present in an amount less than about 10 mole %, preferably less than about 8 mole %, more preferably less than about 5 mole %, more preferably less than or equal to about 4 mole %, more preferably less than about 2 mole %, more preferably less than about 1.5 mole %, more preferably less than about 1 mole %.

The present invention is further directed to a product prepared according to any of the processes described herein. In an embodiment, the present invention is directed to a product prepared according to the process as described in Scheme 1, above. In another embodiment, the present invention is directed to a product prepared according to the process as described in Scheme 2, above.

In another embodiment, the present invention is directed to a product (comprising the compound of formula (I-S)), prepared according to any of the processes as described herein, wherein said product comprises less than about 10 mole %, preferably less than about 8 mole %, more preferably less than about 5 mole %, more preferably less than or equal to about 4 mole %, more preferably less than about 2 mole % of the compound of formula (I-T).

### Synthesis Examples

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### 4-Chloro-1,2-phenylene dibenzoate (4-Chlorocatechol dibenzoate)

[chemical structure]

4-Chlorobenzene-1,2-diol (2.89 g, 20.0 mmol) was dissolved in 25 ml of acetone and 4.55 g (45.0 mmol) of triethylamine and cooled to 5° C. Benzoyl chloride (5.9 g, 42 mmol) was added drop-wise and under stirring within 30 min. Stirring was continued for 30 min at 5° C. Cold water (30 g) was then added within 25 min and stirring was continued for an additional hour. The solid precipitate was collected by filtration and washed with cold water. The residue was dried at 50° C. under full vacuum to yield the title compound (7.0 g, 19.8 mmol/99%) as a white-beige powder.

Purity: >99.0%

### Example 2

### 4-Chloro-1,2-phenylene diacetate (4-Chlorocatechol diacetate)

[chemical structure]

4-Chlorobenzene-1,2-diol (7.23 g, 50.0 mmol) was dissolved in 100 ml of toluene and 11.64 g (115.0 mmol) of triethylamine and cooled to 5° C. Acetyl chloride (8.44 g, 107.5 mmol) was added drop-wise and under vigorous stirring within 30 min. Stirring was continued for 30 min at 5° C. The reaction mixture was quenched with cold water (20 g). The phases were split, and the organic layer was washed with saturated sodium bicarbonate solution (15 ml), followed by a washing with water (15 ml). From the toluene layer, 90 ml were distilled-off at 40° C. under vacuum. Cyclohexane (60 ml) was added and the solution was cooled to 5° C. The resulting mixture was seeded and stirred for 2 h at 5° C. The solid precipitate was collected by filtration and washed with a 75:25 (v/v) mix of cyclehexane/toluene. The residue was dried at 35° C. under full vacuum to yield the title compound (10.2 g, 44.6 mmol/89%) as a white powder.

Purity: >99.0%

### Example 3

### 4-Chloro-1,2-phenylene bis-2,2-dimethylpropanoate (4-Chlorocatechol dipivaloate)

[chemical structure]

4-Chlorobenzene-1,2-diol (723 mg, 5.0 mmol) was dissolved in 250 ml of toluene and 1.21 g (13.0 mmol) of triethylamine and cooled to 5° C. Pivaloyl chloride (1.33 g, 11.0 mmol) was added under stirring within 5 min. Stirring was continued for 30 min at 5° C. The reaction mixture was quenched with cold water (10 g). The aqueous layer was removed, and the organic layer then washed with water (15 ml). The toluene layer was concentrated in vacuum to yield the title compound (4.0 g, 38% (w/w) solution) as a yellow oil which was used in the next step without further purification.

### Example 4

### 4-chloro-1,2-phenylene isobutyl dicarbonate (4-Chlorocatechol di-isopropylcarbonate)

[chemical structure]

4-Chlorobenzene-1,2-diol (4.32 g, 30.0 mmol) was dissolved in 60 ml of tert.-butyl methyl ether (TBME) and 7.08 g (70.0 mmol) of triethylamine and cooled to 5° C. Isobutyl chloroformiate (8.88 g, 65.0 mmol) was added under stirring within 15 min. Stirring was continued for 30 min at 5° C. Cold water (20 g) was added. The aqueous layer was removed, and the organic layer then washed with water (15 ml) and brine (15 ml). The organic layer was evaporated to yield the title compound 9.5 g (quant.) as a colorless oil, which was used in the next step without further purification.

### Example 5

### 4-Chlorocatechol benzoate (Mixture of 5-chloro-2-hydroxyphenyl benzoate and 5-chloro-2-hydroxyphenyl benzoate)

Mixture of

[chemical structure]

4-Chlorobenzene-1,2-diol (2.89 g, 20.0 mmol) and 4-Chloro-1,2-phenylene dibenzoate (7.41 g, 21.0 mmol) were suspended in 25 ml of toluene and 0.4 g (4.0 mmol) of triethylamine. The mixture was heated to 65° C. for 2 h, then cooled to 55° C. and seeded. Stirring was continued for 3 h. Cyclohexane (25 ml) was dosed within 1 h. The resulting solution was gradually cooled to 22° C. within 8 h. The solid precipitate was collected by filtration and washed with a 1.1 (v/v) mixture of toluene/cyclohexane. The residue was dried at 40° C. under full vacuum to yield the title compound (8.7 g, 35.0 mmol/87%) as a white-beige powder.

Purity: >95%

### Example 6

### (S)-(6-chloro-2,3-dihydrobenzo[b][1,4]dioxin-2-yl)methanol

[chemical structure]

4-Chiorocatechol mono benzoate (746 mg, 3.0 mmol) and (R)-glycidyl-3-nitrobenzene sulfonate (661 mg, 2.55 mmol) were dissolved in 7 ml of DMA at 22° C. 1.59 g (7.5 mmol, 2.5 eq) of dry powdered K₃PO₄ was added in one portion. The resulting mixture was stirred for 5 h at room temperature and then diluted with 8 ml of TBME. The suspension was filtered, and the solids washed with TBME. To the filtrate a solution of acetic acid (300 mg, 5 mmol) and sodium chloride (1 g) in water (9 ml) was added under stirring. The phases were split and the organic layer washed with water. A 10 wt-% NaOH solution (15 g, 37.5 mmol) was added, followed by tetrabutylammonium chloride (5 mg) and the biphasic mixture was stirred for 6 h at 22° C. The water layer was removed, the organic layer washed twice with water. The organic layer was evaporated under reduced pressure and the product crystallized from isopropanol/water (1:3 v/v, 6 ml). Filtration and washing of the precipitate yielded the title compound (375 mg) as brownish crystals.

Regio-isomeric ratio (desired:undesired): 89:11.

### Example 7

### (S)-(6-chloro-2,3-dihydrobenzo[b][1,4]dioxin-2-yl)methanol

[chemical structure]

4-Chlorocatechol (434 mg, 3.0 mmol) and 4-Chiorocatechol diacetate (754 mg, 3.3 mmol) were dissolved in DMA (10 ml). K₃PO₄ (1.27 g, 6 mmol) was added and the suspension was stirred for 2 h at 22° C. (R)-glycidyl-3-nitrobenzene sulfonate (1.39 g, 5.4 mmol) was added to the mixture, followed by another portion of K₃PO₄ (1.9 g, 9 mmol) of. The resulting mixture was stirred for 5 h at room temperature. A 10 wt-% NaOH solution (6 g) was added and the mixture was stirred over night. The solids were filtered, the mixture was diluted with water and extracted three times with TBME. The combined organic layers were washed with 10% NaOH solution and water, and dried (Na₂SO₄). Filtration and evaporation of the solvent yielded an oily residue that was crystallized from cyclohexane (5 ml) to yield the title compound as brownish crystal (590 g).

Regio-isomeric ratio (desired:undesired): 91:9.

### Example 8

### (S)-(6-chloro-2,3-dihydrobenzo[b][1,4]dioxin-2-yl)methanol

[chemical structure]

4-Chlorocatechol (434 mg, 3.0 mmol) and 4-Chlorocatechol dipivaloate (1.64 g, 3.1 mmol as 38% w/w solution in toluene) were dissolved in DMA (12 ml). K₃PO₄ (606 g, 3 mmol) was added and the suspension was stirred for 3 h at 22° C. (R)-glycidyl-3-nitrobenzene sulfonate (1.42 g, 5.5 mmol) was added to the mixture, followed by another portion of K₃PO₄ (1.9 g, 9 mmol). The resulting mixture was stirred for 5 h at room temperature, and then diluted with TBME (12 ml). The suspension was filtered, the solids washed with TBME (15 ml). To the filtrate a solution of acetic acid (600 mg, 10 mmol) and sodium chloride (2.5 g) in water (15 ml) was added under stirring. The aqueous layer was removed, and the organic layer was washed with water. The solvent was evaporated under vacuum and the residue dissolved in methanol. A 20% solution of NaOH was added. The solution was stirred at room temperature for 1 h, then warmed to 60° C. and stirred for another hour. Water was added and the clear brown solution cooled to 5° C. and seeded. Precipitation was completed by step-wise addition of water. The solid precipitate was collected by filtration and washed first with a 5:1 (v/v) mixture of water/isopropanol, then with pure water, to yield the title compound as off-white crystals (580 g).

Regio-isomeric ratio (desired:undesired): 92:9

### Example 9

### (S)-(6-chloro-2,3-dihydrobenzo[b][1,4]dioxin-2-yl)methanol

[chemical structure]

4-Chlorocatechol (434 mg, 3.0 mmol) and 4-chloro-1,2-phenylene isobutyl dicarbonate (1.0 g, 3.2 mmol) were dissolved in DMA (12 ml). K₃PO₄ (606 g, 3 mmol) was added and the suspension was stirred for 6 h at 22° C. (R)-glycidyl-3-nitrobenzene sulfonate (1.29 g, 5.0 mmol) was added to the mixture, followed by another portion of K₃PO₄ (1.9 g, 9 mmol). The resulting mixture was stirred for 5 h at room temperature, and then diluted with TBME (12 ml). The suspension was filtered, the solids washed with TBME (15 ml). To the filtrate a solution of acetic acid (600 mg, 10 mmol) and sodium chloride (2.5 g) in water (15 ml) was added under stirring. The aqueous layer was removed, and the organic layer was washed with water. The solvent was evaporated under vacuum and the residue dissolved in methanol. A 20% solution of NaOH in was added. The solution was stirred at room temperature for 1 h, then warmed to 60° C. and stirred for another hour. Water was added and the clear brown solution cooled to 5° C. and seeded. Precipitation was completed by step-wise addition of water. The solid precipitate was collected by filtration and washed first with a 5:1 (v/v) mixture of water/isopropanol, then with pure water, to yield the title compound as off-white crystals.

Regio-isomeric ratio (desired:undesired): 96.2:3.8

### Example 10

### (S)-(6-chloro-2,3-dihydrobenzo[b][1,4]dioxin-2-yl)methanol

[chemical structure]

A stirred suspension of potassium-tert.-butoxide (7.75 g, 69 mmol) in THF (20 ml) was cooled to −25° C. A solution of 4-Chlorocatechol (4.34 g, 30 mmol) in THF (9 ml) was added. The suspension was stirred for 25 min at −25° C. 4-Chloro-1,2-phenylene-diacetate (7.88 g, 34.5 mmol) in THF (9 ml) was added drop-wise, while maintaining the temperature at −20 to −30° C. Stirring was continued for 10 min. The cold, dark-blue mixture was dosed within 10 min to a solution of (R)-Glycidyl-3-nitrobenzenesulfonate (14.1 g, 54.4 mmol) in DMF (75 ml), cooled to 5° C. The resulting mixture was then stirred for 4 h at 0-5° C. The suspension was diluted with TBME (50 ml) and filtered, the filter cake washed with TBME (50 ml). At a temperature of 5° C., acetic acid (0.9 g) was added, followed by a solution of NaCl (18.2 g) in water (82 g). The phases were split and the aqueous layer extracted once more with TBME (50 ml). The combined organic layers were washed with water. A 10 wt-% NaOH solution (32 g) was added, followed by benzyltriethylammonium chloride (0.1 g), and the bi-phasic mixture was stirred for 12 h. The aqueous layer was removed, and the organic layer was washed with a 10 wt % NaOH solution, twice with water and once with brine. The organic phase was stirred with 2.5 g of silica gel for 2 h. The solids were filtered and washed with TBME. The solvent was distilled-off at 45° C. under vacuum and gradually replaced with cyclohexane (total of 100 ml). The resulting suspension was heated to 55° C., seeded and aged for 2 h. The suspension was cooled to 10° C. within 6 h by a cubic cooling curve. Isolation of the product by filtration, washing with cyclohexane and drying yielded the title compound (6.8 g, 43.9 mmol, 62%) as an off-white residue.

Purity >97%; Regio-isomeric ratio of 95.7:4.3.

### Example 11

### (S)-(6-chloro-2,3-dihydrobenzo[b][1,4]dioxin-2-yl)methanol

[chemical structure]

A stirred suspension of potassium-tert-butoxide (4.94 g, 44 mmol) in THF (15 ml) was cooled to −25° C. A solution of 4-Chlorocatechol (2.89 g, 20 mmol) in THF (5 ml) was added. The suspension was stirred for 25 min at −25° C. 4-Chloro-1,2-phenylene-dibenzoate (8.11 g, 23 mmol) in THF (12 ml) was added drop-wise, while maintaining the temperature at −20 to −30° C. Stirring was continued for 30 min. The cold, dark-blue mixture was given to a solution of (R)-Glycidyl-3-nitrobenzenesulfonate (9.47 g, 36.5 mmol) in DMF (45 ml), cooled to 5° C. The resulting mixture was then stirred for 4 h at 0-5° C. A cold 10 wt-% NaOH solution (38.4 g) was added and stirring was continued for 5 h at 0-5° C. The suspension was filtered, and the solids washed with TBME (35 ml). TBME and THF were distilled-off at reduced pressure. Brine (60 g) was added and the water phase was extracted twice with TBME (50 ml). The combined organic layers were washed twice with a 10 wt % NaOH solution (30 g), twice with water (20 g) and once with brine. The organic phase is stirred with silica gel (1 g) for 2 h. The solids were filtered and washed with TBME. The solvent was distilled-off at 45° C. under vacuum and gradually replaced with cyclohexane (total of 130 ml). The resulting suspension was heated to 55° C., seeded and aged for 2 h. The suspension was cooled to 10° C. within 6 h by a cubic cooling curve. Isolation of the product by filtration, washing with cyclohexane and drying yielded the title compound (4.8 g, 23.9 mmol, 65%) as an off-white material.

Purity >97%; Regio-isomeric ratio of 99.1:0.9.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of a compound of formula (I) [chemical structure]
wherein
b is an integer from 0 to 4;
each R⁵ is independently selected from the group consisting of halogen and C₁₋₄alkyl;
and pharmaceutically acceptable salts thereof; comprising [chemical structure]
reacting a mixture of a compound of formula (V) and a compound of formula (VI), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a base; in an organic solvent or mixture of solvents; at a temperature in the range of from about −10° C. to about 25° C.; to yield a mixture of the corresponding compound of formula (VII-a) and the corresponding compound of formula (VII-b), wherein Cation⁺ is the corresponding base cation; [chemical structure]
reacting the mixture of the compound of formula (VII-a) and the compound of formula (VII-b) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-a) and the corresponding compound of formula (VIII-b); [chemical structure]
reacting the mixture of the compound of formula (VIII-a) and the compound of formula (VIII-b) with an aqueous base or a mixture of an aqueous base and an alcohol; in an organic solvent or mixture of organic solvents; at a temperature in the range of from about −10° C. to about 25° C.; to yield the corresponding compound of formula (I).

2. A process as in claim 1, [chemical structure]
is 2-(6-chloro-2,3-dihydro-benzo[1,4]dioxinyl) and a is 1.

3. A process for the preparation of compounds of formula (I) [chemical structure]
wherein
b is an integer from 0 to 4;
each R⁵ is independently selected from the group consisting of halogen and C₁₋₄alkyl;
and pharmaceutically acceptable salts thereof; comprising [chemical structure]
reacting a mixture of a compound of formula (V) and a compound of formula (VI), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a phosphate; in an organic solvent or mixture of solvents; at about room temperature; to yield a mixture of the corresponding compound of formula (VII-a) and the corresponding compound of formula (VII-b), wherein Cation⁺ is the corresponding phosphate cation; [chemical structure]
reacting the mixture of the compound of formula (VII-a) and the compound of formula (VII-b) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-a) and the corresponding compound of formula (VIII-b); [chemical structure]
reacting the mixture of the compound of formula (VIII-a) and the compound of formula (VIII-b) with an aqueous base or a mixture of an aqueous base and an alcohol; in an organic solvent or mixture of organic solvents; at a temperature in the range of from about −10° C. to about 25° C.; to yield the corresponding compound of formula (I).

4. A process as in claim 3, [chemical structure]
is 2-(6-chloro-2,3-dihydro-benzo[1,4]dioxinyl) and a is 1.

5. A process for the preparation of a compound of formula (I-S) [chemical structure]
or a pharmaceutically acceptable salt thereof; comprising [chemical structure]
reacting a mixture of a compound of formula (V-S) and a compound of formula (VI-S), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a base; in an organic solvent or mixture of solvents; at a temperature in the range of from about −10° C. to about 25° C.; to yield a mixture of the corresponding compound of formula (VI-Sa) and the corresponding compound of formula (VII-Sb), wherein Cation⁺ is the corresponding base cation; [chemical structure]
reacting the mixture of the compound of formula (VII-Sa) and the compound of formula (VII-Sb) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-Sa) and the corresponding compound of formula (VIII-Sb); [chemical structure]
reacting the mixture of the compound of formula (VIII-Sa) and the compound of formula (VIII-Sb) with an aqueous base or a mixture of an aqueous base and an alcohol; in an organic solvent or mixture of organic solvents; at a temperature in the range of from about −10° C. to about 25° C.; to yield the corresponding compound of formula (I-S).

6. A process as in claim 5, wherein —C(O)—R¹ is acyl; and wherein the acyl is selected from the group consisting of acetyl, pivaloyl and benzoyl.

7. A process as in claim 6, wherein the acyl is benzoyl.

8. A process as in claim 5, wherein the compound of formula (VI-S) is present in an amount in the range of from about 1.0 to about 1.5 molar equivalents.

9. A process as in claim 8, wherein the compound of formula (VI-S) is present in an amount in the range of from about 1.1 to about 1.2 molar equivalents.

10. A process as in claim 5, wherein the base is selected from the group consisting of DBU, KOtBu, LiOtBu, NaOC₅H₁₁ and Cs₂CO₃.

11. A process as in claim 10, wherein the base is selected from the group consisting of DBU, KOtB and LiOtBu.

12. A process as in claim 11, wherein the base is KOtBu.

13. A process as in claim 5, wherein the base is present in an amount in the range of from about 1.0 to about 3.0 molar equivalents.

14. A process as in claim 12, wherein the base is present in an amount in the range of from about 2.1 to about 2.5 molar equivalents.

15. A process as in claim 5, wherein the mixture of the compound of formula (V-S) and the compound of formula (VI-S) is reacted at a temperature in the range of from about −25° C.

16. A process as in claim 5, wherein the compound of formula (IX) is selected from the group consisting of (R)-glycidyl-3-nitrobenzenesulfonate, (R)-glycidyl-4-nitrobenzenesulfonate and (R)-glycidyl-4-tolylsulfonate.

17. A process as in claim 16, wherein the compound of formula (IX) is (R)-glycidyl-3-nitrobenzensulfonate.

18. A process as in claim 5, wherein the compound of formula (IX) is present in an amount in the range of from about 0.2 to about 3.0 molar equivalents.

19. A process as in claim 18, wherein the compound of formula (IX) is present in an amount of about 0.8 to about 1.0 molar equivalents.

20. A process as in claim 5, wherein the aqueous base is selected from the group consisting of NaOH, KOH and LiOH.

21. A process as in claim 20, wherein the aqueous base is NaOH.

22. A process as in claim 5, wherein the aqueous base is present in an amount in the range of from about 2.0 to about 8.0 molar equivalents.

23. A process as in claim 22, wherein the aqueous base is present in an amount of about 4.0 molar equivalents.

24. A process as in claim 5, wherein the mixture of the compound of formula (VIII-Sa) and the compound of formula (VIII-Sb) is reacted at a temperature in the range of from about 0° C. to about 5° C.

25. A product prepared according to the process of claim 5.

26. A process for the preparation of a compound of formula (I-S) [chemical structure]
or a pharmaceutically acceptable salt thereof; comprising [chemical structure]
reacting a mixture of a compound of formula (V-S) and a compound of formula (VI-S), wherein R¹ is selected from the group consisting of C₁₋₄alkyl, C₂₋₄alkenyl, substituted aryl, aralkyl, heteroaryl, —O—C₁₋₄alkyl, —O—C₂₋₄alkenyl, —O— (substituted aryl), —O-aralkyl and —O-heteroaryl, with a phosphate; at about room temperature; to yield a mixture of the corresponding compound of formula (VI-Sa) and the corresponding compound of formula (VII-Sb), wherein Cation⁺ is the corresponding phosphate cation; [chemical structure]
reacting the mixture of the compound of formula (VII-Sa) and the compound of formula (VII-Sb) with a compound of formula (IX), an (R)-glycidyl-(substituted aryl)sulfonate wherein R² is selected from the group consisting of hydrogen, halogen, methyl, ethyl, nitro, cyano and dichloro; to yield a mixture of the corresponding compound of formula (VIII-Sa) and the corresponding compound of formula (VIII-Sb); [chemical structure]
reacting the mixture of the compound of formula (VIII-Sa) and the compound of formula (VIII-Sb) with an aqueous base or a mixture of an aqueous base and an alcohol; in an organic solvent or mixture of organic solvents; at a temperature in the range of from about −10° C. to about 25° C.; to yield the corresponding compound of formula (I-S).

27. A process as in claim 26, wherein the phosphate is selected from the group consisting of potassium phosphate and sodium phosphate.

28. A process as in claim 26, wherein the phosphate is present in an amount in the range of from about 2.0 to about 10.0 molar equivalents.

29. A process as in claim 26, wherein the phosphate is potassium phosphate; and wherein the potassium phosphate is present in an amount of about 6.0 molar equivalents.

30. A product prepared according to the process of claim 26.

31. A product prepared as in claim 5, comprising less than about 10 mole % of the compound of formula (I-T)

[chemical structure]

32. A product prepared as in claim 5, comprising less than about 5 mole % of the compound of formula (I-T) [chemical structure]
